(19) United States
(12) Patent Application Publication
(10) Pub. No.: US 2009/0169645 A1
(43) Pub. Date: Jul. 2, 2009

(54) Iron Sulfate-Based Phosphate Adsorbent

(75) Inventors: Hans-Martin Muller, Engelburg (CH); Erik Philipp, Wittenbach (CH); Peter Geisser, St. Gallen (CH)

Correspondence Address: RANKIN, HILL & CLARK LLP, 925 EUCLID AVENUE, SUITE 700, CLEVELAND, OH, 44115-1405, US

(73) Assignee: Vifor (International) AG, St. Gallen (CH)

(21) Appl. No.: 11/571,305

(22) PCT Filed: Jun. 21, 2005
(86) PCT No.: PCT/EP05/52861

§ 371 Date: Feb. 6, 2007

(30) Foreign Application Priority Data
Jun. 28, 2004 (DE) 10 2004 031 181.1

(51) Int. Cl. A61K 33/26

(52) U.S. Cl. 424/646

(57) ABSTRACT

The present invention relates to a process for the preparation of a novel composition, and to the use of the composition as a phosphate adsorbent, in particular for administration in humans or animals.

The present invention relates to a process for the preparation of a novel composition, and to the use of the composition as a phosphate adsorbent, in particular for administration in humans or animals.

An adsorbent for phosphate from aqueous media is known from EP 0 868 125, which adsorbent contains polynuclear β-iron hydroxide stabilised by carbohydrates and/or humic acid. The product is produced by reacting an iron(III) chloride solution with a base (in particular soda solution) and adding the carbohydrate or humic acid before the resulting iron hydroxide ages. The use of the iron(III) chloride solution in the precipitation is necessary because the presence of chloride ions is essential for the formation of the β-iron hydroxide (akaganeite). It is assumed that the addition of the carbohydrate or humic acid causes stabilisation of the freshly prepared β-iron hydroxide, as a result of which the resulting material exhibits a superior phosphate-absorbing capacity as compared with a mixture of aged β-iron hydroxide with carbohydrates or humic acid.

However, the use of iron(III) chloride as the starting material in the preparation of the phosphate adsorbent according to EP 0 868 125 gives rise to problems. For example, the use of iron(III) chloride leads to corrosion problems in the installation owing to the presence of chloride ions. In addition, the cost of iron(III) chloride is relatively high.

It was, therefore, desirable to provide a phosphate adsorbent that does not exhibit the above-described disadvantages owing to the use of iron(III) chloride as starting material. At the same time, the phosphate adsorbent should have substantially the same phosphate-adsorbing capacity as the material according to EP 0 868 125.

The present inventors have now found, surprisingly, that by using iron sulfate and/or iron nitrate compounds as starting material it is possible, without using iron(III) chloride, to obtain an iron hydroxide which is evidently likewise stabilised, for example, by carbohydrates or humic acids and whose phosphate-adsorbing capacity corresponds substantially to that of the material of EP 0 868 125. On the basis of this finding, the inventors completed the present patent application.

The invention accordingly provides a process for the preparation of a composition, comprising the following steps:
a) adding at least one base to an aqueous, sulfate- and/or nitrate-containing iron(III) salt solution to form a precipitate of iron hydroxide,
b) optionally washing the resulting precipitate one or more times with water, yielding an aqueous suspension of the iron hydroxide,
c) adding to the resulting aqueous suspension at least one further constituent that inhibits ageing of the precipitate of iron hydroxide obtained in step b),
d) drying the composition obtained in step c).

In step a), an aqueous, sulfate- and/or nitrate-containing iron(III) salt solution is reacted with at least one base to form a precipitate of iron hydroxide.

The aqueous, sulfate-containing iron(III) salt solution may be in particular a solution of iron(III) sulfate (Fe₂(SO₄)₃) (including its hydrates) in water. It is also possible, however, to use other aqueous, sulfate-containing iron(III) salt solutions, such as solutions of iron alums, such as KFe(SO₄)₂ or NH₄Fe(SO₄)₂. It is further possible according to the invention to use sulfuric-acid-containing solutions of iron(II) sulfate, which are subjected to oxidation, for example with nitric acid.

The aqueous, sulfate-containing iron(III) salt solution that is used preferably has a concentration of approximately from 3 to 16 wt. %, based on the amount of iron.

The aqueous, nitrate-containing iron(III) salt solution may be in particular a solution of iron(III) nitrate (Fe(NO₃)₃) (including its hydrates) in water.

The aqueous, nitrate-containing iron(III) salt solution that is used preferably has a concentration of approximately from 3 to 16 wt. %, based on the amount of iron.

The amount of base added in step a) is expediently so chosen that a pH value of at least about 3, preferably of at least about 6, is established. Expediently, the amount of base used is such that, from an economic point of view, the iron precipitates from the solution as completely as possible. In general, therefore, the procedure is carried out at pH values of not more than about 10. Higher pH values are no longer expedient from an economic point of view. The pH value established in step a) is, therefore, preferably approximately from 3 to 10, more preferably approximately from 5 to 8.

There is preferably used as the base in step a) an alkali metal and/or alkaline earth metal compound. Such compounds are particularly preferably hydroxides or carbonates of alkali or alkaline earth metals. Alkali carbonates, alkali bicarbonates and alkali metal hydroxides, especially of sodium, are more preferred. The bases are expediently and preferably used in the form of an aqueous solution, preferably having a molarity of approximately from 0.01 to 2 mol/l. It is, however, also possible to add the bases in solid form to the sulfate- and/or nitrate-containing iron(III) salt solution.

There is most preferably used as the base in step a) sodium hydroxide, sodium carbonate and/or sodium bicarbonate, preferably in the form of their aqueous solutions.

The reaction with the base is preferably not carried out at elevated temperatures, because these might lead to accelerated ageing of the hydroxide that is formed. The temperature in the reaction is preferably maintained in the range from 10 to 40° C., more preferably from 20 to 30° C.; even more preferably, the reaction is carried out at room temperature (25° C.). The suspension can expediently be allowed to rest for a short time after the precipitation. In practice, the suspension can be left to stand, for example, for from 1 to 5 hours at room temperature or below. During that time, the suspension can be stirred.

The resulting precipitate is then preferably washed once, preferably several times, with water, the water being removed after the washing/suspension operation in each case preferably by decanting, filtering, centrifugation and/or by processes of reverse osmosis, for example by membrane filtration. The resulting moist product is not dried. The moist product is suspended in water. The amount of water is not critical; preferably, the procedure is such that the iron content of the resulting suspension (calculated as Fe) is up to 10 wt. %, particularly preferably from 2 to 8 wt. %.

The resulting aqueous suspension of the iron hydroxide preferably has an approximately neutral pH value in the range of approximately from 6.5 to 7.5, before the further constituent is added. Lower pH values would result in the iron hydroxide going partly into solution again. Higher pH values are undesirable because they can lead to complex formation in step c).

The process according to the invention is particularly preferably carried out in such a manner that substantially no ageing of the iron hydroxide has occurred before the addition of the further constituent in step c). During the ageing of precipitates, the re-grouping of initially randomly placed molecules to form a more or less regular crystal lattice often takes place. The ageing of precipitates in most cases involves not only crystallisation but also particle enlargement as a result of Ostwald ripening.

In step c) there is added to the suspension obtained above at least one further constituent that inhibits the above-described ageing of the precipitate of iron hydroxide obtained in step b). This constituent inhibiting ageing of the iron hydroxide can preferably be selected from the group consisting of carbohydrates, carbohydrate derivatives and humic acid. The constituent is preferably added in solid form, but addition in the form of an aqueous solution is also possible in principle.

According to the invention there are particularly preferably used as the further ageing-inhibiting constituent carbohydrates, such as various carbohydrates and sugars, for example agarose, dextran, dextrin, maltodextrin, dextrin derivatives, dextran derivatives, starch, cellulose, such as microcrystalline cellulose and cellulose derivatives, sucrose, maltose, lactose or mannitol.

Particular preference is given to starch, sucrose, dextrin and/or a mixture thereof. Starch, sucrose or a mixture thereof are most preferred. A mixture of sucrose and at least one further constituent selected in particular from starch, maltodextrin and cellulose, especially microcrystalline cellulose, is very preferred. The function of the additional constituent is presumably—without being bound to one theory—to be regarded as that of stabilising the freshly precipitated iron hydroxide, whereby ageing of the iron hydroxide precipitate is prevented.

It is preferable to select the amount of carbohydrates or humic acid so that at least 0.5 g, preferably at least 1 g, of the further constituent inhibiting ageing of the iron hydroxide, such as carbohydrate and/or humic acid, is added per g of iron (calculated as Fe). Preferably, the iron content of the resulting composition should be not more than 50 wt. %, preferably not more than about 40 wt. %. The iron content of the resulting composition should preferably be at least 20 wt. %. The maximum content of the constituent inhibiting ageing of the iron hydroxide, such as carbohydrates and/or humic acid, is not subject to any limitation and is determined primarily by economic reasons. The mentioned content is preferably approximately from 5 to 60 wt. %, more preferably approximately from 20 to 60 wt. %.

After the addition in step c) of the constituent inhibiting ageing of the iron hydroxide, the resulting aqueous suspension is dried in a manner known per se. The drying can be carried out, for example, by concentration in vacuo or by spray drying.

In a preferred embodiment of the process according to the invention, at least one calcium salt is added before or after the composition obtained according to the invention is dried. Suitable calcium salts are, for example, salts of inorganic or organic acids, in particular calcium acetate. The addition of the calcium salt increases the phosphate-binding capacity, especially at higher pH values. It is particularly advantageous to use such adsorbents provided with calcium salts at pH values of more than 5, because even then the complete phosphate-binding capacity is retained. It has been shown that an addition of from 400 mg to 2 g, for example about 1 g, of calcium salt, especially calcium acetate, per g of iron is particularly advantageous.

The material obtained according to the invention is substantially a physical mixture of iron hydroxide and the constituent inhibiting ageing of the iron hydroxide, such as carbohydrates or humic acid. As already mentioned above, it is assumed that the latter come into contact with the freshly precipitated iron hydroxide and lead to stabilisation of the iron hydroxide, so that no ageing of the material, which reduces the phosphate-adsorbing ability, occurs. Complex formation, as described in DE 42 39 442, cannot occur under the conditions chosen according to the invention of the addition of an aqueous suspension, because complex formation requires strongly alkaline conditions during the addition of, for example, carbohydrates to the iron hydroxide.

The compositions obtained by the process according to the invention are preferably used in the production of an adsorbent for phosphate from aqueous solutions. Preferably, a preparation for oral and/or parenteral administration in humans or animals is produced from the compositions obtained by the process according to the invention. In particular, the compositions obtained according to the invention are used in the production of a preparation for the prophylaxis and/or treatment of the hyperphosphataemic state. Particularly preferably, the compositions obtained according to the invention are used in the production of a preparation for the prophylaxis and/or treatment of dialysis patients.

To that end, the compositions obtained according to the invention are formulated in a manner known per se into pharmaceutical dosage forms, such as, for example, for oral administration. They can be formulated as such or together with conventional pharmaceutical additives, such as conventional carriers or auxiliary substances. For example, encapsulation can be carried out, it being possible to use as encapsulating agents conventional materials used in the pharmaceutical sector, such as hard or soft gelatin capsules. Microencapsulation of the compositions obtained according to the invention is also possible. It is also possible to provide the adsorbents, optionally together with auxiliary substances and additives, in the form of granules, tablets, dragées, filled into sachets, in gel form or in the form of sticks. The daily dose of the compositions obtained according to the invention is, for example, from 1 to 3 g, preferably approximately 1.5 g, based on iron.

The compositions obtained according to the invention are also suitable for use for the adsorption of phosphate bonded to foodstuffs; for this purpose they may be mixed into foodstuffs, for example. To that end there may be prepared, for example, formulations as described above for medicaments.

The compositions obtained according to the invention are suitable in particular as adsorbents especially for inorganic and foodstuffs-bonded phosphate from body fluids, chyme and foodstuffs. They have a phosphate-adsorbing ability similar to that of the agents obtained according to EP 0868125 and can be produced simply and inexpensively.

The invention relates further to an adsorbent obtained by the process according to the invention.

By using iron sulfate and/or iron nitrate as starting material it is possible according to the invention to obtain a composition having a particularly low content of chloride, which is present in the composition only in traces. The chloride content is especially lower than the chloride content conventional for akaganeite. The invention accordingly relates also to a composition containing iron(III) hydroxide as well as at least one constituent selected from the group consisting of carbohydrates and humic acid, which composition contains less than 0.05 wt. %, preferably less than 0.03 wt. %, more preferably less than 0.01 wt. %, chloride.

The invention is explained in greater detail by means of the following examples:

### EXAMPLE 1

444 g of iron(III) sulfate solution (11.3% w/w Fe) are added dropwise in the course of 20-30 minutes, with stirring (vane-type stirrer), to 1160 g of soda solution (d²⁰1.185 g/ml). The suspension is stirred for a further one hour. Thereafter, 2 litres of water are added to the suspension, with stirring; the mixture is left to stand and then the supernatant solution is decanted off. This procedure is repeated five times. In this manner, 1238 g of a suspension having an iron content of 4.0% (w/w) (determined by complexometry) are obtained. 73.9 g of each of sucrose and starch are added to the 1238 g of the above suspension. The suspension is then concentrated in a rotary evaporator at 60° C. and dried under a high vacuum at 50° C. 223 g of powder having an iron content of 21.5% (w/w) are obtained.

Determination of the phosphate-adsorbing capacity: 10 ml of sodium phosphate solution (13.68 g/l Na₃PO₄×12 H₂O) are added to 233 mg of the material prepared according to the above Example (corresponding to 0.9 mmol of iron) (molar ratio Fe:P=1:0.4). After adjustment of the pH value, the suspension is allowed to react at 37° C. for 2 hours. The suspension is then centrifuged; the supernatant is decanted off and made up to 25 ml with distilled water, and its phosphorus content is determined. The phosphate adsorption of the material prepared according to the Example, determined by ion chromatography, was 0.20 mg P/mg Fe at a pH of 3.0 and 0.16 mg P/mg Fe at a pH of 5.5.

### EXAMPLE 2

439 g of iron(III) sulfate solution (11.5% w/w Fe) are added dropwise in the course of 20-30 minutes, with stirring (vane-type stirrer), to 1014 ml of sodium hydroxide solution (9.6% w/v). The suspension is stirred for a further one hour. Thereafter, 2 litres of water are added to the suspension, with stirring; the mixture is left to stand and the supernatant solution is then decanted off. This procedure is repeated until the supernatant that is decanted off is free of sulfate (control with barium chloride). In this manner, 1606 g of a suspension having an iron content of 2.74% (w/w) (determined by complexometry) are obtained. 66.0 g of each of sucrose and starch are added to the 1606 g of the above suspension. The suspension is then concentrated in a rotary evaporator at 60° C. and dried under a high vacuum at 50° C. 190 g of powder having an iron content of 22.2% (w/w) are obtained.

Determination of the phosphate-adsorbing capacity: 10 ml of sodium phosphate solution (13.68 g/l Na₃PO₄×12 H₂O) are added to 226 mg of the material prepared according to the Example (corresponding to 0.9 mmol of iron) (molar ratio Fe:P 1:0.4). After adjustment of the pH value, the suspension is allowed to react at 37° C. for 2 hours. The suspension is then centrifuged; the supernatant is decanted off and made up to 25 ml with distilled water, and its phosphorus content is determined.

The phosphate adsorption of the material prepared according to Example 1, determined by ion chromatography, was 0.19 mg P/mg Fe at a pH of 3.0 and 0.15 mg P/mg Fe at a pH of 5.5.

### EXAMPLE 3

535 g of iron(III) nitrate solution (9.7% w/w Fe) are added dropwise in the course of 20-30 minutes, with stirring (vane-type stirrer), to 1200 g of soda solution (d²⁰=1.185 g/ml). The suspension is stirred for a further one hour. The suspension is then transferred to a filter bag and washed for 3 hours by continuously rinsing with water (conductivity of the washing water after 3 hours about 300 μS/cm). In this manner, 923 g of a suspension having an iron content of 4.3% (w/w) (determined by complexometry) are obtained. 60.1 g of each of sucrose and starch are added to the 923 g of the above suspension. The suspension is then concentrated in a rotary evaporator at 60° C. and dried under a high vacuum at 50° C. 172 g of powder having an iron content of 22.3% (w/w) are obtained.

Determination of the phosphate-adsorbing capacity: 10 ml of sodium phosphate solution (13.68 g/l Na₃PO₄×12 H₂O) are added to 225 mg of the material prepared according to the Example (corresponding to 0.9 mmol of iron) (molar ratio Fe:P=1:0.4). After adjustment of the pH value, the suspension is allowed to react at 37° C. for 2 hours. The suspension is then centrifuged; the supernatant is decanted off and made up to 25 ml with distilled water, and its phosphorus content is determined. The phosphate adsorption of the material prepared according to the Example, determined by ion chromatography, was 0.21 mg P/mg Fe at a pH of 3.0 and 0.17 mg P/mg Fe at a pH of 5.5.

### EXAMPLE 4

234 g of iron(III) sulfate solution (11.4% w/w Fe) are added dropwise in the course of 20-30 minutes, with stirring (vane-type stirrer), to 615 g of soda solution (d²⁰=1.185 g/ml). The suspension is stirred for a further one hour. The suspension is then transferred to a filter bag and washed for about 3 hours by continuously rinsing with water (test for absence of sulfate with barium chloride). In this manner, 470 g of a suspension having an iron content of 6.0% (w/w) (determined by complexometry) are obtained. 21.1 g of each of sucrose and maltodextrin are added to the 470 g of the above suspension. The suspension is then concentrated in a rotary evaporator at 60° C. and dried under a high vacuum at 50° C. 66 g of powder having an iron content of 20.3% (w/w) are obtained.

Determination of the phosphate-adsorbing capacity: 10 ml of sodium phosphate solution (13.68 g/l Na₃PO₄×12 H₂O) are added to 247 mg of the material prepared according to the Example (corresponding to 0.9 mmol of iron) (molar ratio Fe:P=1:0.4). After adjustment of the pH value, the suspension is allowed to react at 37° C. for 2 hours. The suspension is then centrifuged; the supernatant is decanted off and made up to 25 ml with distilled water, and its phosphorus content is determined. The phosphate adsorption of the material prepared according to the Example, determined by ion chromatography, was 0.21 mg P/mg Fe at a pH of 3.0 and 0.17 mg P/mg Fe at a pH of 5.5.

### EXAMPLE 5

223 g of iron(III) sulfate solution (11.3% w/w Fe) are added dropwise in the course of 20-30 minutes, with stirring (vane-type stirrer), to 585 g of soda solution (d²⁰=1.185 g/ml). The suspension is stirred for a further one hour. The suspension is then transferred to a filter bag and washed for about 3 hours by continuously rinsing with water (test for absence of sulfate with barium chloride). In this manner, 447 g of a suspension having an iron content of 6.0% (w/w) (determined by complexometry) are obtained. 20.6 g of each of sucrose and crystalline cellulose are added to the 447 g of the above suspension. The suspension is then concentrated in a rotary evaporator at 60° C. and dried under a high vacuum at 50° C. 65 g of powder having an iron content of 20.6% (w/w) are obtained.

Determination of the phosphate-adsorbing capacity: 10 ml of sodium phosphate solution (13.68 g/l Na3PO₄×12 H₂O) are added to 244 mg of the material prepared according to the Example (corresponding to 0.9 mmol of iron) (molar ratio Fe:P=1:0.4). After adjustment of the pH value, the suspension is allowed to react at 37° C. for 2 hours. The suspension is then centrifuged; the supernatant is decanted off and made up to 25 ml with distilled water, and its phosphorus content is determined. The phosphate adsorption of the material prepared according to the Example, determined by ion chromatography, was 0.20 mg P/mg Fe at a pH of 3.0 and 0.17 mg P/mg Fe at a pH of 5.5.

1. A process to prepare a composition, comprising:
(a) adding at least one base to an aqueous, sulfate- and/or nitrate-containing iron(III) salt solution to form a precipitate of iron hydroxide,
(b) optionally washing the resulting precipitate one or more times with water, yielding an aqueous suspension of the iron hydroxide,
(c) contacting the aqueous suspension and at least one constituent that inhibits ageing of the iron hydroxide,
(d) drying the composition obtained in step (c).

2. The process of claim 1 wherein a sulfate-containing iron (III) salt solution is used.

3. The process of claim 1, wherein the constituent inhibiting ageing of the precipitate of iron hydroxide is selected from the group consisting of carbohydrates, carbohydrate derivatives and humic acid.

4. The process of claim 1, wherein the iron(III) salt solution is an aqueous solution of iron(III) sulfate.

5. The process of claim 1, wherein the iron(III) salt solution is an aqueous solution of iron(III) nitrate.

6. The process of claim 1, wherein a pH value of at least 3 is established in step a).

7. The process of claim 1, wherein the resulting composition contains not more than 50 wt. % iron.

8. The process of claim 1, wherein the base is selected from alkali metal and alkaline earth metal compounds.

9. The process of claim 1, wherein the base is selected from alkali metal hydroxides and alkali metal carbonates.

10. The process of claim 1, wherein an aqueous solution of an alkali metal carbonate, of an alkali metal bicarbonate or of an alkali metal hydroxide is used as the base in step a).

11. The process of claim 1, wherein a pH value of at least 6 is established in step a).

12. The process of claim 1, wherein sodium hydroxide, sodium carbonate and/or sodium bicarbonate is used as the base in step a).

13. The process of claim 1, wherein the constituent inhibiting ageing of the iron hydroxide is selected from the group consisting of starch, sucrose and dextrin.

14. The process of claim 1, wherein at least one calcium salt is added before or after the composition is dried.

15. The process of claim 1, wherein substantially no ageing of the iron hydroxide has occurred before the addition of the constituent in step c).

16. The process of claim 1, wherein the resulting composition is formulated in a further step e) into a pharmaceutical dosage form.

17. Use of the composition obtainable according to claim 1 in the production of an adsorbent for phosphate from aqueous solutions.

18. Use of the composition obtainable according to claim 1 in the production of a preparation for oral and/or parenteral administration in humans or animals.

19. Use of the composition obtainable according to claim 1 in the production of a preparation for the prophylaxis and/or treatment of hyperphosphatemia.

20. Use according to claim 17 in the production of a preparation for the prophylactic and/or therapeutic treatment of dialysis patients.

21. An adsorbent comprising the composition made by the process of claim 1.

22. Use of the composition obtained according to claim 1 in the production of an adsorbent for mixing with foodstuffs.

23. A composition comprising iron(III) hydroxide and at least one selected from the group consisting of carbohydrates and humic acid, wherein the composition contains less than 0.05 wt. % chloride.
